(19) United States
(12) Patent Application Publication
(10) Pub. No.: US 2026/0324969 A1
(43) Pub. Date: Oct. 22, 2026

(54) Methods and Systems for the Diagnosis of Cataracts, Light Scattering, and Refractive Errors in Human Eyes

(71) Applicants: Andrew Xiao Liang, Fremont, CA (US); Ling Yu, Fremont, CA (US); Junzhong Liang, Fremont, CA (US)

(72) Inventors: Andrew Xiao Liang, Fremont, CA (US); Ling Yu, Fremont, CA (US); Junzhong Liang, Fremont, CA (US)

(21) Appl. No.: 18/865,878

(22) PCT Filed: May 22, 2023
(86) PCT No.: PCT/US23/67285

§ 371 Date: Nov. 14, 2024

Related U.S. Application Data
(60) Provisional application No. 63/473,387, filed on May 24, 2022.
(60) Provisional application No. 63/473,511, filed on Jun. 6, 2022.

(51) Int. Cl. A61B 3/117; A61B 3/028; A61B 3/10; A61B 3/14

(52) U.S. Cl. CPC A61B 3/1176; A61B 3/0285; A61B 3/1015; A61B 3/14

(57) ABSTRACT

Methods and systems are disclosed for obtaining a see-through diagnosis of human eyes by 1) delivering a narrow light beam into an eye to form a compact spot of light at the retina, 2) using the sensor module to detect the light reflected from the retina across the pupil, 3) providing a see-through diagnosis using the detected light distribution across the pupil for an early detection of cataracts, tracking cataract development, and monitoring post-op refractive surgeries. The narrow light beam delivered into the eye can be positioned inside the pupil to avoid being blocked by cataracts. The see-through methods and systems can be further configured 1) to provide an objective measurement of eye's aberrations by using a lenslet array or a plurality of gratings together integrated with an image sensor as the sensor module, 2) for measuring diffusion of light in the eye using eye's double-pass point-spread distributions.

### BACKGROUND

A cataract is a progressive, painless clouding of the natural, internal lens of the eye. Cataracts block light, making it difficult to see clearly. Over an extended period of time, cataracts can cause blindness. They're often related to growing older, but sometimes they can develop in younger people.

Cataracts are a common vision problem, particularly among senior citizens. By age 75, APPROXIMATELY HALF OF ALL AMERICANS HAVE CATARACTS. About 20 million people worldwide are blind due to cataracts.

Cataracts are diagnosed in a compreshensive eye exam. Ophthamlogists and optometrists will take a medical history and perform a series of eye tests including a visual acuity test for measuring the eyesight in eye, a retinal exam for examing eye's retina, and a slit-lamp exam for a magnified view of eye's lens, iris, cornea and the other structures at the front of your eye.

Cataracts can be observed, characterized, and graded by ophthamlogists and optometrists in the Slit-Lamp Exam. In some cases, photographic images of cataracts are taken from patient's front view, shown in FIG. 1, as a part of medical record.

Several deficiencies are found in today's comprehensive eye exams, particularly for cataract care, and they include: 1) providing effective and proper refraction for eyes with cataracts before a surgery intervention, 2) objectively quantifying and tracking cataract development to determine the best time for a cataract surgery with an IOL implant, and 3) early detection of cataracts.

Consequently, although many configurations and methods are known in the art, these conventional methods and systems suffer from one or more disadvantages.

### SUMMARY

In some embodiments, we describe a method for obtaining a see-through diagnosis of an eye, comprising the steps of: delivering a light beam into an eye, wherein the light beam forms a compact spot of light at the retina of the eye; using an optical relay to reproduce the light reflected from the retina to a sensor module; using the sensor module to detect the light reflected from the retina, passing through the crystalline lens and the cornea, and forming a reflected beam across the pupil of the eye; providing a see-through diagnosis of the eye using the detected light distribution across the pupil.

In some additional embodiments, we disclose a method for measuring wave aberrations of an eye with cataracts, comprising the steps of: delivering a light beam into an eye, wherein the light beam forms a compact spot of light at the retina of the eye; using an optical relay to reproduce the light reflected from the retina to a Hartmann-Shack sensor module; using the lenslet array together with the image sensor of the Hartmann-Shack sensor to detect the light reflected from the retina and forming a reflected beam across the pupil of the eye, wherein the lens array forms an array of focus spots on the image sensor; performing a wavefront analysis using an algorithm for the detection of not only the focusing spots of light passing through pupil area without a cataract but also the missing focusing spots blocked by cataracts in the Hartmann-Shack sensor; providing wave aberration for the eye with cataracts from the focusing spots of light passing through pupil area without a cataract as well as the missing focusing spots blocked by cataracts in the Hartmann-Shack sensor.

In some additional embodiments, we describe a system for providing a see-through diagnosis of an eye, comprising: a light source module for delivering a light beam into an eye, wherein the light beam forms a compact spot of light at the retina of the eye; an optical relay module for reproducing the light reflected from the retina to a sensor module; a sensor module to detect light reflected from the retina, passing through the crystalline lens and cornea, and forming a reflected beam across the pupil of the eye; an output module for providing a see-through diagnosis of an eye using the detected light distribution across the pupil.

In still some additional embodiments, we describe a system for measuring wave aberration of an eye with cataracts, comprising: a light source module for delivering a light beam into an eye, wherein the light beam forms a compact spot of light at the retina of the eye; an optical relay module to reproduce the light reflected from the retina to a Hartmann-Shack sensor module; a Hartmann-Shack sensor module with a lenslet array and an image sensor to detect the light reflected from the retina which forms a reflected beam across the pupil of the eye, wherein the lens array forms an array of focus spots on the image sensor; a software module for performing a wavefront analysis using an algorithm for the detection of not only the focusing spots of light passing through pupil area without a cataract but also the missing focusing spots blocked by cataracts in the Hartmann-Shack sensor; a wavefront reconstruction software module for providing wave aberration for the eye with cataracts from the focusing spots of light passing through pupil area without a cataract as well as the missing focusing spots blocked by cataracts in the Hartmann-Shack sensor; a processor module for controlling hardware modules and software modules.

In some embodiment, we describe a method for obtaining a see-through diagnosis of an eye, comprising the steps of: delivering a narrow light beam into an eye through the cornea and the crystalline lens and the light beam forming a compact spot of light at the retina, wherein the narrow light beam has an effective dimension less than 1.5 mm in diameter and can be positioned to various locations across the pupil of the eye in order to avoid being blocked by eye conditions such as cataracts; using a sensor module to detect the light reflected from the retina, which then passes through the crystalline lens and the cornea and forms a reflected beam across the pupil of the eye, wherein the sensor module has at least an image sensor; providing a see-through diagnosis of the eye using the detected light distribution across the pupil.

In some embodiment, we describe a system for obtaining a see-through diagnosis of an eye, comprising, a light source module for delivering a narrow light beam into an eye through the cornea and the crystalline lens and the light beam forming a compact spot of light at the retina, wherein the narrow light beam has an effective dimension less than 1.5 mm in diameter and can be positioned to various locations across pupil of the eye in order to avoid being blocked by eye conditions such as cataracts; a sensor module to detect light reflected from the retina, which then passes through the crystalline lens and cornea and forms a reflected beam across the pupil of the eye; an output module for providing a see-through diagnosis of an eye using the detected light distribution across the pupil.

### BRIEF DESCRIPTION OF THE DRAWINGS

FIG. 1 shows some photographic images of cataracts taken from patient's front view.

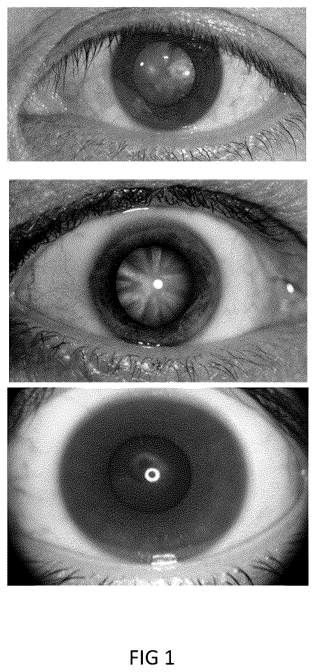

FIG. 2 schematically illustrates the principle for taking a see-through image of a human body using X-rays.

FIG. 3 schematically illustrates the principle for taking a see-through image of eye's optics in some embodiments according to the present invention.

FIG. 4 shows photographic images of some eyes with cataracts taken in eye exams using eye's see-through principal in FIG. 3.

FIG. 5 shows photographic images of some real eyes taken in eye exams for post-op refractive surgery using eye's see-through principal in FIG. 3.

FIG. 6 schematically illustrates the principle for measuring light scattering in eyes using a double-pass point-spread distribution.

FIG. 7 schematically illustrates the principle for taking a see-through image of eye's optics according to some embodiments of the present invention.

### DETAILED DESCRIPTION

Reference now will be made in detail to embodiments of the disclosed invention, one or more examples of which are illustrated in the accompanying drawings. Each example is provided by way of explanation of the present technology, not as a limitation of the present technology. In fact, it will be apparent to those skilled in the art that modifications and variations can be made in the present technology without departing from the spirit and scope thereof. For instance, features illustrated or described as part of one embodiment may be used with another embodiment to yield a still further embodiment. Thus, it is intended that the present subject matter covers such modifications and variations as come within the scope of the appended claims and their equivalents.

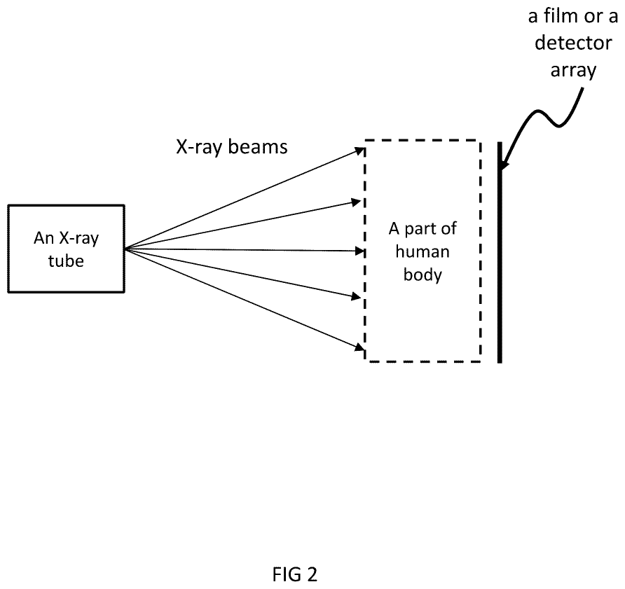

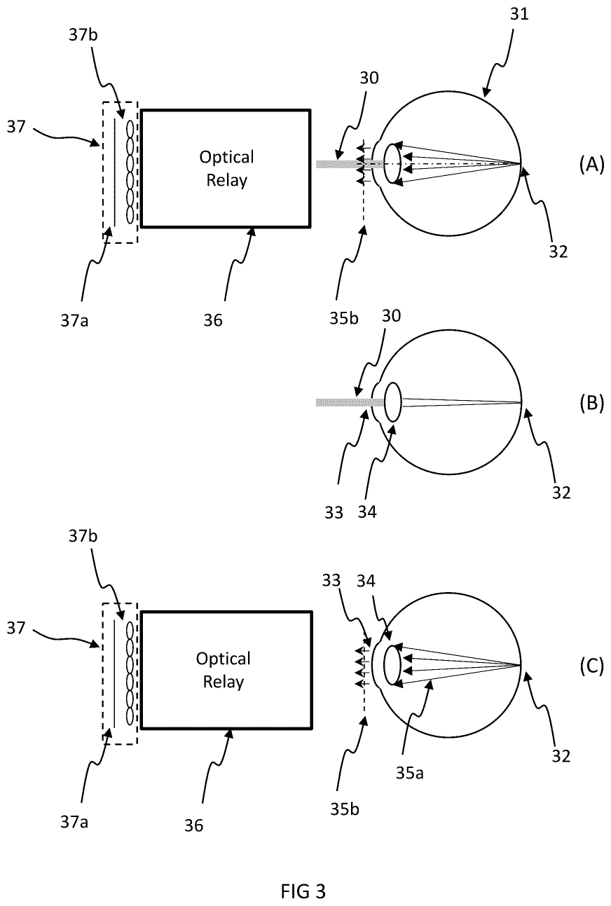

The X-ray machine is considered one of the most important inventions in medicine. Bone X-ray uses a very small dose of X-ray beam, schematically illustrated in FIG. 2, to produce pictures of any bone in the body using a film or a detector array (a see-through view of human bodies), and is commonly used to diagnose fractured bones or joint dislocation. We cannot get a see-though view of cataracts similarly because it is impossible to place an image sensor inside a human eye noninvasively. Instead, we propose a method in FIG. 3A to obtain a see-through view of cataracts.

In some embodiments, the see-through method in FIG. 3 comprises the steps of: 1) delivering a light beam 30 into an eye 31, which forms a compact spot of light at retina of the eye 32, which is also shown in FIG. 3B separately for an incoming pass through cornea 33 and crystalline lens 34, 2) detecting the reflected light 35b in the return pass from the eye, which is also shown in FIG. 3C separately, generated from the reflected light 35a from retina 32 and passing-through the entire crystalline lens 34 and the partial cornea 33 due to an iris between the crystalline lens and the cornea, 3) using an optical relay 36 to reproduce the pass-through beam 35b to a sensor module (37), 4) providing a see-through diagnosis using the detected light distribution across the pupil.

In one embodiment, the sensor module consists of only an image sensor 37a. In another embodiment, the sensor module consists of an image sensor 37a and a lenslet array 37b. In yet another embodiment, the image sensor 37a is placed at the focal point of the lenslet array 37b in order to increase efficiency for detecting the pass-through beam 35b.

In one embodiment, the light beam delivered into the eye has a small beam profile so that the beam can be positioned at different locations inside eye's pupil into avoid being blocked by eye conditions such as cataracts. p Preferably, the small beam profile has an effective dimension less than 1.5 mm in diameter.

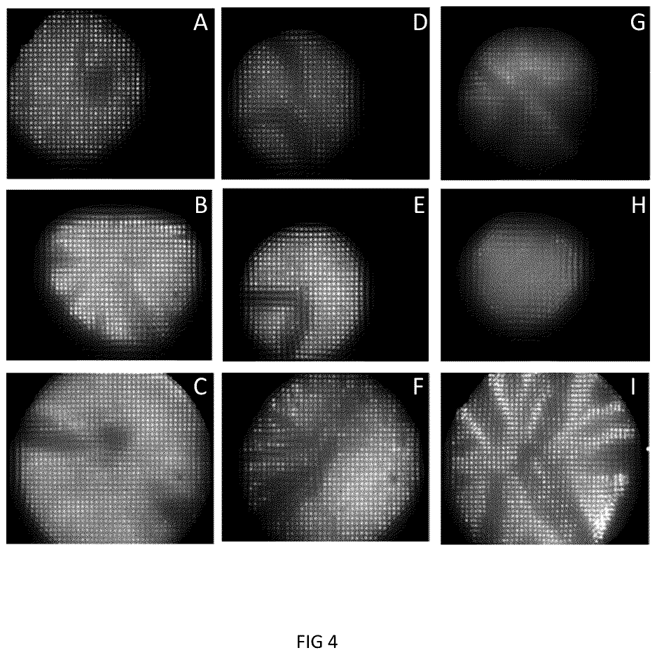

In FIG. 4, we show see-through images of 9 eyes with cataracts in an experimental system built according the see-through principle in FIG3 with the following characteristics: 1) the probing beam 30 is infrared light (840 nm wavelength, less than 100 microwatts) and has an effective beam dimension less than 1 mm in diameter, and the narrow probing beam was moved within the eye's pupil so that it could be focused on to the eye's retina without being blocked by cataracts in the eyes, 2) the sensor module has a 2-dimensional lenslet array of 10 m by 10 mm and 0.15 mm from lenslet to lenslet, and an image sensor for the detection of the light reflected light from eye's retina and passing-through through crystalline lenses and cornea, 3) the optical relay is a pair of lenses that reproduce the pass-through beam 35b to the plane of the lenslet array.

It is obvious that the see-through images for cataracts in FIG. 4 can lead to the diagnosis of: 1) cataracts of different types based on the dark patterns of light across the pupil that was blocked by cataracts, 2) various stages of cataracts in development, 3) severity of cataracts in picture G with missing the lower half portion, in Picture H with missing most of the central portion, in picture I with severe cortical cataracts throughout of the pupil.

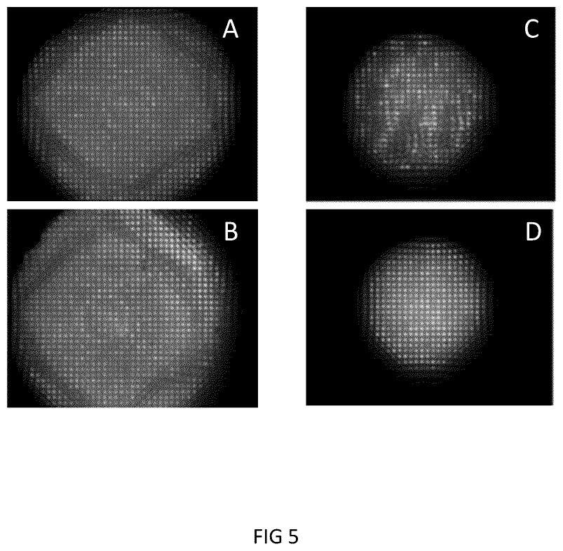

In FIG. 5, we show see-through images of 4 eyes for post-op refractive surgeries using the same experimental setup as for FIG. 4 and according to the see-through principle in FIG 3. Observation of the see-through images leads to the diagnosis of the eyes for 1) corneal surgeries shown by the dark marks in the see-through view In Picture A and Picture B, 2) healing of an eye one week after a refractive surgery in Picture C and in 6 months in D, which explained poor visual acuity for the eye in an one week post-op exam.

In one embodiment, the see-through diagnosis can be used for early detection of cataracts, tracking cataract development, making decision for surgery intervention for cataracts, monitoring a healing process for a post-op refractive surgery, and qualification of dry eyes.

In another embodiment, the see-through diagnosis further includes an analysis that determines a pattern for cataracts in an eye, and using the pattern to generate its diffraction/scattering of light so that glare, halo, and ghost images in eyes can be diagnosed.

In one embodiment, the method of see-through diagnosis in FIG. 3 can be further configured to provide wavefront measurement of the tested eye using the lens array and the image sensor in FIG. 3 as a Hartmann-Shack wavefront sensor, as invented and first described by Liang, Grimm, Goelz and Bille in “Objective measurement of wave aberrations of the human eye with the use of a Hartmann-Shack wave-front sensor” in Journal of the Optical Society of America A Vol. 11, Issue 7, pp. 1949-1957 (1994).

For the wavefront analysis of eyes with cataracts, an advanced algorithm was invented for the detection of not only the focusing spots of light passing through the pupil area without a cataract but also the missing focusing spots blocked by cataracts in the Hartmann-Shack test. In one embodiment, the advanced algorithm for performing the wavefront analysis comprises: providing an estimate of the distance between the bright focusing spots of light passing through pupil area without a cataract, localizing the bright focusing spots of light passing through the pupil area without a cataract, finding the missing focusing spots blocked by cataracts in the Hartmann-Shack sensor.

It is advantageous to use one single system shown in FIG. 3 in the present invention to provide simultaneously: 1) measurements of an eye's aberrations, 2) see-through view for cataracts and post-op refractive surgeries.

In another embodiment, the described wavefront measurement is further configured to determine at least an objective sphero-cylindrical correction, and the sphero-cylindrical correction consists of an objective spherical power (SPH_o), an objective cylinder power (CYL_o), and an objective cylinder axis (AXIS_o).

In yet one embodiment, as described by Junzhong Liang and Ling Yu in “Methods and systems for optimizing refractive refraction of human eyes” in PCT/US22/26459, the sphero-cylindrical correction for the described wavefront measurements is further configured to provide a range for the objective cylinder power (CYL_o) for at least some eyes or include a quality metrics for at least one of 1) measuring the confidence level in the objectively determined cylinder power and cylinder axis in addition to the objective sphero-cylinder correction, 2) assessing/displaying quality of vision corrections for a plurality of cylinder power.

In one embodiment, the see-through methods in FIG. 3 for simultaneous see-through imaging of cataracts and wavefront measurement of human eye is further configured include optical diagnosis of the eye based on eye's wave aberration. The optical diagnosis includes at least one of 1) specifying other aberrations beyond the objective sphero-cylinder correction such as coma, spherical aberration, 2) providing an acuity diagnosis, 3) providing objective evaluation of eye's optical quality including but not limited to a point-spread function or a Strehl ratio, a MTF (Modulation-Transfer Function).

In one embodiment, the see-through method in FIG. 3 for simultaneous see-through imaging of cataracts as well as wavefront measurement is further configured to use a phoropter module to determine a subjectively modified prescription.

In another embodiment, the see-through method further includes selling eyeglasses using subjectively modified prescription.

In some embodiments, we describe a system for providing a see-through diagnosis of an eye. It comprises of: 1) a light source module for delivering a light beam into an eye, and the light beam forms a compact spot of light at the retina of the eye, 2) an optical relay module for reproducing the light reflected from the retina to a sensor module; 3) a sensor module to detect light reflected from the retina, passing through the crystalline lens and cornea, and forming a return beam across the pupil of the eye; 4) an output module for providing a see-through diagnosis according to the detected light distribution across the pupil.

In one embodiment, the see-through diagnosis can be used for an early detection of cataracts, tracking cataract development, monitoring a healing process for a post-op refractive surgery, and qualification for dry eyes.

In another embodiment, the see-through diagnosis further includes determining a pattern for cataracts in an eye, and using the determined cataract pattern to generate diffraction/scattering of light for the diagnosis of glare, halo, and ghost images.

In one embodiment, the light beam delivered into the eye in the see-through system has a small beam profile so that the beam can be positioned at various locations across pupil in order to avoid being blocked by cataracts or other eye conditions. In another embodiment, the small beam profile has an effective dimension less than 1.5 mm in diameter.

In one embodiment, the sensor module consists of an image sensor. In another embodiment, the sensor module consists of an image sensor and a lenslet array. In yet another embodiment, the image sensor is placed at the focal point of the lenslet array in order to increase efficiency for detecting the pass-through beam.

In one embodiment, the system for see-through view is further configured to provide wavefront measurement of the tested eye using the lens array and the image sensor as a Hartmann-Shack sensor.

In another embodiment, the system is further configured to use the wavefront measurement to determine at least an objective sphero-cylindrical correction, and the sphero-cylindrical correction consists of an objective spherical power (SPH_o), an objective cylinder power (CYL_o), and an objective cylinder axis (AXIS_o).

In yet another embodiment, the sphero-cylindrical correction is further configured to provide a range for the objective cylinder power (CYL_o) for at least some eyes, or include a quality metrics for at least one of i) measuring the confidence level in the objectively determined cylinder power and cylinder axis in addition to the objective sphero-cylinder correction, ii)assessing/displaying quality of vision corrections for a plurality of cylinder powers In still another embodiment, the system for see-through view with wavefront measurement further includes optical diagnosis of the eye based on eye's wave aberration. The optical diagnose includes at least one of i) specifying other aberrations beyond the objective sphero-cylinder correction such as coma, spherical aberration, ii)providing an acuity diagnosis, iii) providing objective evaluation of eye's optical quality including but not limited to a point-spread function or a Strehl ratio, a MTF (Modulation Transfer Function).

In one embodiment, the system for see-through view and wavefront measurements is further configured to use a phoropter module to determine a subjectively modified prescription.

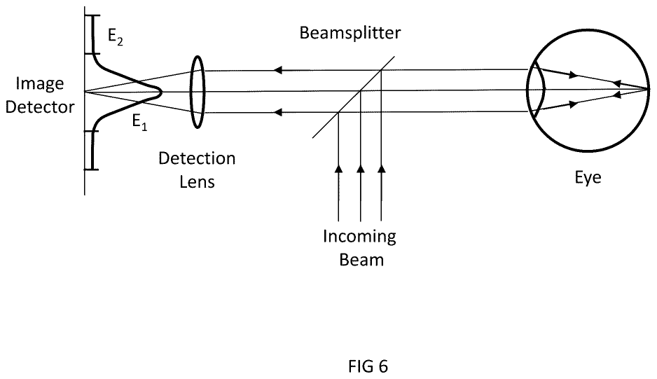

In addition to cataracts and refractive errors, diffusion of light is another factor that affects human vision. Diffusion of light is associated with glare at night, and it has been subjectively measured since 1920s, including by Holiday in 1927 and by Stiles in 1929. The first objective measurement of light diffusing for humans was reported for studying light diffusion for aged eyes from a double-pass point-spread function of eyes by Westheimer and Liang in “Evaluating diffusion of light in the eye by objective means” Investigative Ophthalmology & Visual Science, Vol. 35, No. 5, pp 2652-2657 as illustrated in FIG. 6. A beam of light is focused by the eye's optics onto the retina, and the reflected light is brought, via a beamsplitter, to a focus on the light detecting device. The retinal image of a point light source is spread out over an area larger than the geometric image, an effect has roots in a variety of causes, including scatter in the ocular media, optical aberrations, and diffraction of light. An index of diffusion was developed that describes the relative spread of light inside and outside the region of image focus, providing a measure of light scatter.

In one embodiment, the system for see-through view and wavefront measurements further includes a double-pass module for measuring light diffusion in the human eyes, which forms a combined 3-element system for the diagnosis of cataracts, light diffusing, and refractive errors. The double-pass module delivers a light beam and produce a point-spread distribution at the eye's retina (the 1st pass) and detects the light reflected from the retina that is focused on to an image sensor (the 2nd pass). The light diffusion in the human eye is measured by a relative ratio of the light energy in a first zone covering the pedestal of the eye's double-pass point-spread distribution, which is primarily caused by light scattering in eye's optics, to the light energy in a second zone in the center of the eye's double-pass point-spread distribution, which is primarily contributed by the image blur due to aberrations in the eye and light diffraction.

In another embodiment, the combined 3-element system further includes a module for the correction of an eye's focus errors (myopia or hyperopia) and astigmatism (a cylinder power with a cylinder axis).

In yet another embodiment, the combined 3-element system further includes optical components to reduce corneal reflection in obtaining the double-pass point-spread distribution.

In still another embodiment, the combined, 3-element system further includes a software model for deriving eye's MTF from the double-pass measurement.

In some embodiments, we describe a method for measuring wave aberration of an eye with cataracts, and it comprises the steps of: a) delivering a light beam into an eye, which forms a compact spot of light at retina of the eye, b) using an optical relay to reproduce the light reflected from the retina to a Hartmann-Shack sensor module, c) using the lenslet array together with the image sensor of the Hartmann-Shack sensor to detect the light reflected from the retina and forming a reflected beam across the pupil of the eye while the lens array forms an array of focus spots on the image sensor; d) performing a wavefront analysis using an algorithm for the detection of not only the focusing spots of light passing through pupil area without a cataract but also the missing focusing spots blocked by cataracts in the Hartmann-Shack sensor; e) providing wavefront aberrations for the eye with cataracts from the focusing spots of light passing through pupil area without a cataract as well as the missing focusing spots blocked by cataracts in the Hartmann-Shack sensor.

In one embodiment, performing the wavefront analysis for eyes with cataracts comprises: a) providing an estimate of the distance between the bright focusing spots of light passing through pupil area without a cataract; b) localizing the bright focusing spots of light passing through pupil area without a cataract; c) finding the missing focusing spots blocked by cataracts in the Hartmann-Shack sensor.

In another embodiment, the method for wavefront measurement of eyes with cataracts is further configured to use the wavefront measurement to determine at least an objective sphero-cylindrical correction, and the sphero-cylindrical correction consists of an objective spherical power (SPH_o), an objective cylinder power (CYL_o), and an objective cylinder axis (AXIS_o)

In another embodiment, the sphero-cylindrical correction for the eyes with cataracts is further configured to provide a range for the objective cylinder power (CYL_o) for at least some eyes or include a quality metrics for at least one of i) measuring the confidence level in the objectively determined cylinder power and cylinder axis in addition to the objective sphero-cylinder correction, ii) assessing/displaying quality of vision corrections for a plurality of cylinder power.

In yet another embodiment, the method for wavefront measurement of eyes with cataracts is further configured to use a phoropter module to determine a subjectively modified prescription.

In some embodiment, we describe a system for measuring wave aberration of an eye with cataracts, and the system comprises: 1) a light source module for delivering a light beam into an eye which forms a compact spot of light at retina of the eye, 2) an optical relay module to reproduce the light reflected from the retina to a detection plane, 3) a Hartmann-Shack sensor module with a lenslet array and an image sensor placed at the detection plane in order to detect the light reflected from the retina which forms a reflected beam across the pupil of the eye, and the lens array forms an array of focus spots on the image sensor, 4) a software module for performing a wavefront analysis using an algorithm for the detection of not only the focusing spots of light passing through pupil area without a cataract but also the missing focusing spots blocked by cataracts in the Hartmann-Shack sensor, 5) a wavefront reconstruction software module for providing wave aberration for the eye with a cataract from the focusing spots of light passing through pupil area without a cataract as well as the missing focusing spots blocked by cataracts in the Hartmann-Shack sensor, 6) a processor module for controlling hardware modules and software modules.

In one embodiment, performing the wavefront analysis in a system for measuring wave aberration of an eye with cataracts comprises: a) providing an estimate of the distance between the bright focusing spots of light passing through the pupil area without a cataract, b) detecting the bright focusing spots of light passing through pupil area without a cataract, c) estimating the focusing spots blocked by cataracts in the Hartmann-Shack sensor.

In another embodiment, the system for measuring wave aberration of an eye with cataracts is further configured to use wave aberration of an eye to determine at least an objective sphero-cylindrical correction, and the sphero-cylindrical correction consists of an objective spherical power (SPH_o), an objective cylinder power (CYL_o), and an objective cylinder axis (AXIS_o), In yet another embodiment, the sphero-cylindrical correction is further configured to provide a range for the objective cylinder power (CYL_o) for at least some eyes or include a quality metrics for at least one of i) measuring the confidence level in the objectively determined cylinder power and cylinder axis in addition to the objective sphero-cylinder correction, ii)assessing/displaying quality of vision corrections for a plurality of cylinder power.

In still another embodiment, the system for measuring wave aberration of an eye with cataracts is further configured to use a phoropter module to determine a subjectively modified prescription.

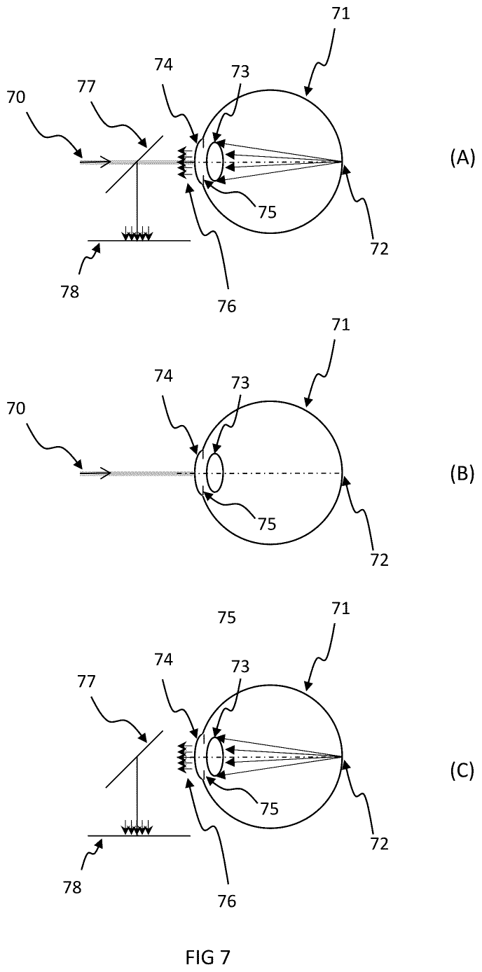

In some embodiment, we describe a method for obtaining a see-through diagnosis of an eye as shown in FIG. 7A. The method comprises the steps of: 1) delivering a narrow light beam 70 into an eye 71 through the cornea 74 and the crystalline lens 73 and the light beam forming a compact spot of light at the retina 72, 2) using a sensor module 78 to detect the light reflected from the retina, which passes through the crystalline lens 73 and the cornea 74 and forms a reflected beam 76 across the pupil that is delimited and restricted by the iris 75 of the eye, and the sensor 78 module having at least an image sensor; 3) providing a see-through diagnosis of the eye using the detected light distribution across the pupil. The narrow light beam 70 has an effective dimension less than 1.5 mm in diameter and can be positioned to various locations across the pupil, delimited and restricted by the iris by the iris 75 of the eye, in order to avoid being blocked by eye conditions such as cataracts. A beamsplitter 77 can be used to divide the incoming pass of the probing light into the eye in FIG. 7B and the return pass of light reflected from the retina in FIG. 7C.

In one embodiment, the see-though method diagnosis of the eye in FIG. 7 is used for an early detection of cataracts, tracking cataract development, monitoring a healing process for a post-op refractive surgery, and qualification of dry eyes.

In another embodiment, the see-through diagnosis in FIG. 7 further includes determining a pattern for cataracts in an eye and using the determined cataract pattern to generate diffraction and/or scattering of light for the diagnosis of glare, halo, and ghost images.

In yet another embodiment, the sensor module in the see-through diagnosis in FIG. 7 is further configured for obtaining a wavefront measurement of the eye, and the sensor module further includes a lenslet array or a plurality of gratings as described by Nakano and Murata in “Talbot interferometry for measuring the focal length of a lens” Applied. Optics, Vol. 24, Issue 19, pp. 3162-3166 (1985).

In one embodiment, the delivered narrow light beam into an eye is focused at a position near the cornea with an effective beam profile less than 1 mm in diameter, and projected onto the retina.

In some embodiment, we describe a system for obtaining a see-through diagnosis of an eye shown in FIG. 7A. The system comprises 1) a light source module for delivering a narrow light beam 70 into an eye through the cornea 74 and the crystalline lens 73 and the light beam forming a compact spot of light at the retina 72, 2) a sensor module 78 to detect light reflected from the retina 72, which passes through the crystalline lens 73 and cornea 74 and forms a reflected beam across the pupil that is delimited or restricted by the iris 75 of the eye, 3) an output module for providing a see-through diagnosis of an eye using the detected light distribution across the pupil. The narrow light beam 70 has an effective dimension less than 1.5 mm in diameter and can be positioned to various locations across pupil, delimited or restricted by the iris 75 of the eye, in order to avoid being blocked by eye conditions such as cataracts. A beamsplitter 77 can be used to divide the incoming pass of the probing light into the eye in FIG. 7B and the return pass of light reflected from the retina in FIG. 7C.

In one embodiment, the system for the see-through diagnosis in FIG. 7A is used for early detection of cataracts, tracking cataract development, monitoring a healing process for a post-op refractive surgery, and qualification of dry eyes.

In another embodiment, the sensor module of the system for the see-through diagnosis in FIG. 7A is further configured for obtaining wavefront measurement of the eye, and the sensor module further includes a) a lenslet array, b) a plurality of gratings.

In one embodiment, the delivered narrow light beam into an eye is focused at a position near the cornea with an effective beam profile less than 1 mm in diameter, and projected onto the retina.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed is:

1. A method for obtaining a see-through diagnosis of an eye, comprising the steps of:
delivering a light beam into an eye, wherein the light beam forms a compact spot of light at retina of the eye;
using an optical relay to reproduce the light reflected from the retina to a sensor module;
using the sensor module to detect the light reflected from the retina, passing through the crystalline lens and the cornea, and forming a reflected beam across the pupil of the eye
providing a see-through diagnosis of the eye using the detected light distribution across the pupil.

2. The method of claim 1 wherein the see-through diagnosis is used for early detection of cataracts, tracking cataract development, monitoring a healing process for a post-op refractive surgery, and qualification of dry eyes.

3. The method of claim 1 wherein the see-through diagnosis includes determining a pattern for cataracts in an eye and using the determined cataract pattern to generate diffraction and/or scattering of light for the diagnosis of glare, halo, and ghost images.

4. The method of claim 1, wherein the light beam delivered into the eye has a small beam profile so that the beam can be positioned to various locations across the pupil in order to avoid being blocked by eye conditions such as cataracts in an eye.

5. The method of claim 4, wherein the small beam profile has an effective dimension less than 1.5 mm in diameter.

6. The method of claim 1, wherein the sensor module consists of an image sensor.

7. The method of claim 6 further includes a lenslet array.

8. The method of claim 7 wherein the image sensor is placed at the focal point of the lenslet array.

9. The method of claim 7 further configured to provide wavefront measurement of the tested eye using the lens array and the image sensor as a Hartmann-Shack sensor.

10. The method of claim 9 further configured to use the wavefront measurement to determine at least an objective sphero-cylindrical correction, wherein the sphero-cylindrical correction consists of an objective spherical power (SPH_o), an objective cylinder power (CYL_o), and an objective cylinder axis (AXIS_o).

11. The method of claim 10 wherein the sphero-cylindrical correction is further configured to provide a range for the objective cylinder power (CYL_o) for at least some eyes or include a quality metrics for at least one of i) measuring the confidence level in the objectively determined cylinder power and cylinder axis in addition to the objective sphero-cylinder correction, ii) assessing/displaying quality of vision corrections for a plurality of cylinder power.

12. The method of claim 10 further configured to use a phoropter module to determine a subjectively modified prescription.

13. A method for measuring wave aberration of an eye with cataracts, comprising the steps of:
delivering a light beam into an eye, wherein the light beam forms a compact spot of light at retina of the eye;
using an optical relay to reproduce the light reflected from the retina to a Hartmann-Shack sensor module;
using the lenslet array together with the image sensor of the Hartmann-Shack sensor to detect the light reflected from the retina and forming a reflected beam across the pupil of the eye, wherein the lens array forms an array of focus spots on the image sensor;
performing a wavefront analysis using an algorithm for the detection of not only the focusing spots of light passing through pupil area without a cataract but also the missing focusing spots blocked by cataracts in the Hartmann-Shack sensor;
providing wave aberration for the eye with cataracts from the focusing spots of light passing through pupil area without a cataract as well as the missing focusing spots blocked by cataracts in the Hartmann-Shack sensor.

14. The method of claim 13 wherein performing the wavefront analysis, comprising:
providing an estimate of the distance between the bright focusing spots of light passing through pupil area without a cataract;
localizing the bright focusing spots of light passing through pupil area without a cataract;
finding the missing focusing spots blocked by cataracts in the Hartmann-Shack sensor.

15. The method of claim 13 is further configured to use the wavefront measurement to determine at least an objective sphero-cylindrical correction, wherein the sphero-cylindrical correction consists of an objective spherical power (SPH_o), an objective cylinder power (CYL_o), and an objective cylinder axis (AXIS_o)

16. The method of claim 15 wherein the sphero-cylindrical correction is further configured to provide a range for the objective cylinder power (CYL_o) for at least some eyes or include a quality metrics for at least one of i) measuring the confidence level in the objectively determined cylinder power and cylinder axis in addition to the objective sphero-cylinder correction, ii) assessing/displaying quality of vision corrections for a plurality of cylinder power.

17. The method of claims 15 is further configured to use a phoropter module to determine a subjectively modified prescription.

18. A system for providing a see-through diagnosis of an eye, comprising:
a light source module for delivering a light beam into an eye, wherein the light beam forms a compact spot of light at the retina of the eye;
an optical relay module for reproducing the light reflected from the retina to a sensor module;
a sensor module to detect light reflected from the retina, passing through the crystalline lens and cornea, and forming a reflected beam across the pupil of the eye;
an output module for providing a see-through diagnosis of an eye using the detected light distribution across the pupil.

19. The system of claim 18 wherein the see-through diagnosis is used for an early detection of cataracts, tracking cataract development, monitoring a healing process for a post-op refractive surgery, and qualification of dry eyes.

20. The system of claim 18 wherein the see-through diagnosis includes determining a pattern for cataracts in an eye and using the determined cataract pattern to generate diffraction/scattering of light for the diagnosis of glare, halo, and ghost images.

21. The system of claim 18, wherein the light beam delivered into the eye has a small beam profile so that the beam can be positioned at various locations across the pupil in order to avoid being blocked by eye conditions such as cataracts.

22. The system of claim 18, wherein the small beam profile has an effective dimension less than 1.5 mm in diameter.

23. The system of claim 18, wherein the sensor module consists of an image sensor.

24. The system of claim 23 further includes a lenslet array.

25. The system of claim 23 wherein the image sensor is placed at the focal point of the lenslet array.

26. The system of claim 24 and 25 are further configured to provide wavefront measurement of the tested eye using the lens array and the image sensor as a Hartmann-Shack sensor.

27. The system of claim 26 is future configured to use the wavefront measurement to determine at least an objective sphero-cylindrical correction, wherein the sphero-cylindrical correction consists of an objective spherical power (SPH_o), an objective cylinder power (CYL_o), and an objective cylinder axis (AXIS_o).

28. The system of claim 27 wherein the sphero-cylindrical correction is further configured to provide a range for the objective cylinder power (CYL_o) for at least some eyes, or include a quality metrics for at least one of i) measuring the confidence level in the objectively determined cylinder power and cylinder axis in addition to the objective sphero-cylinder correction, ii)assessing/displaying quality of vision corrections for a plurality of cylinder powers

29. The system of claim 27 and 28 are further configured to use a phoropter module to determine a subjectively modified prescription.

30. The system of claim 27 is further include optical diagnosis of the eye based on the measured wave aberration, wherein the optical diagnose include at least one of i) specifying other aberrations beyond the objective sphero-cylinder correction such as coma, spherical aberration, ii) providing an acuity diagnosis, iii) providing objective evaluation of eye's optical quality including but not limited to a point-spread function or a Strehl ratio, a MTF (Modulation Transfer Function).

31. The system of claim 18 further includes a double-pass module for measuring light diffusion in an eye, wherein the double-pass module delivers a light beam and produce a point-spread distribution at the eye's retina (the 1^{st} pass) and detects the light reflected from the retina that is focused on to an image sensor (the 2^{nd} pass), and wherein the light diffusion in the eye is measured by a relative ratio of the light energy in a first zone covering the pedestal of the eye's double-pass point-spread distribution that is caused by light scattering in eye's optics, to the light energy in a second zone in the center of the eye's double-pass point-spread distribution that is primarily contributed by the image blur due to aberrations in the eye and light diffraction.

32. The system of claim 31 further includes a module for the correction of eye's focus errors (myopia or hyperopia) and astigmatism (a cylinder power with a cylinder axis).

33. The system of claim 31 further includes optical components to reduce corneal reflection in obtaining the double-pass point-spread distribution.

34. The system of claim 31 further includes a software model for deriving eye's MTF from the double-pass measurement.

35. A system for measuring wave aberration of an eye with cataracts, comprising:
a light source module for delivering a light beam into an eye, wherein the light beam forms a compact spot of light at retina of the eye;
an optical relay module to reproduce the light reflected from the retina to a Hartmann-Shack sensor module;
a Hartmann-Shack sensor module with a lenslet array and an image sensor to detect the light reflected from the retina which forms a reflected beam across the pupil of the eye, wherein the lens array forms an array of focus spots on the image sensor;
a software module for performing a wavefront analysis using an algorithm for the detection of not only the focusing spots of light passing through pupil area without a cataract but also the missing focusing spots blocked by cataract in the Hartmann-Shack sensor;
a wavefront reconstruction software module for providing wave aberration for the eye with cataracts from the focusing spots of light passing through pupil area without a cataract as well as the missing focusing spots blocked by cataracts in the Hartmann-Shack sensor a processor module for controlling hardware modules and software modules.

36. The system of claim 35 wherein performing the wavefront analysis comprise:
providing an estimate of the distance between the bright focusing spots of light passing through pupil area without a cataract;
detecting the bright focusing spots of light passing through pupil area without cataracts;
estimating the focusing spots blocked by cataracts in the Hartmann-Shack sensor.

37. The system of claim 35 is further configured to use wave aberration of an eye to determine at least an objective sphero-cylindrical correction, wherein the sphero-cylindrical correction consists of an objective spherical power (SPH_o), an objective cylinder power (CYL_o), and an objective cylinder axis (AXIS_o).

38. The system of 37 wherein the sphero-cylindrical correction is further configured to provide a range for the objective cylinder power (CYL_o) for at least some eyes or include a quality metrics for at least one of i) measuring the confidence level in the objectively determined cylinder power and cylinder axis in addition to the objective sphero-cylinder correction, ii) assessing/displaying quality of vision corrections for a plurality of cylinder power.

39. The system of claim 35 is further configured to use a phoropter module to determine a subjectively modified prescription.

40. A method for obtaining a see-through diagnosis of an eye, comprising the steps of:
delivering a narrow light beam into an eye through the cornea and the crystalline lens and the light beam forming a compact spot of light at the retina, wherein the narrow light beam has an effective dimension less than 1.5 mm in diameter and can be positioned to various locations across the pupil of the eye in order to avoid being blocked by eye conditions such as cataracts;
using a sensor module to detect the light reflected from the retina, which then passes through the crystalline lens and the cornea and forms a reflected beam across the pupil of the eye, wherein the sensor module has at least an image sensor;
providing a see-through diagnosis of the eye using the detected light distribution across the pupil.

41. The method of claim 40 wherein the see-through diagnosis is used for early detection of cataracts, tracking cataract development, monitoring a healing process for a post-op refractive surgery, and qualification of dry eyes.

42. The method of claim 40 wherein the see-through diagnosis includes determining a pattern for cataracts in an eye and using the determined cataract pattern to generate diffraction and/or scattering of light for the diagnosis of glare, halo, and ghost images.

43. The method of claim 40 wherein the sensor module is further configured for obtaining a wavefront measurement of the eye, wherein the sensor module further includes a lenslet array or a plurality of gratings.

44. The method of claim 40 wherein the delivered narrow light beam into an eye is focused at a position near the cornea with an effective beam profile less than 1 mm in diameter, and projected onto the retina.

45. A system for obtaining a see-through diagnosis of an eye, comprising,
a light source module for delivering a narrow light beam into an eye through the cornea and the crystalline lens and the light beam forming a compact spot of light at the retina, wherein the narrow light beam has an effective dimension less than 1.5 mm in diameter and can be positioned to various locations across pupil of the eye in order to avoid being blocked by eye conditions such as cataracts;
a sensor module to detect light reflected from the retina, which then passes through the crystalline lens and cornea and forms a reflected beam across the pupil of the eye;
an output module for providing a see-through diagnosis of an eye using the detected light distribution across the pupil.

46. The system of claim 45 wherein the see-through diagnosis is used for early detection of cataracts, tracking cataract development, monitoring a healing process for a post-op refractive surgery, and qualification of dry eyes.

47. The system of claim 45 wherein the sensor module is further configured for obtaining wavefront measurement of the eye, wherein the sensor module further includes a) a lenslet array, b) a plurality of gratings.

48. The system of claim 45 wherein the delivered narrow light beam into an eye is focused at a position near the cornea with an effective beam profile less than 1 mm in diameter, and projected onto the retina.
